(19) United States
(12) Patent Application Publication
(10) Pub. No.: US 2012/0045833 A1
(43) Pub. Date: Feb. 23, 2012

(54) Bioreactor, control system and control method thereof

(76) Inventors: Yi Gao, Guangdong (CN); Huangcheng Zhou, Guangdong (CN); Yan Wang, Guangdong (CN); Xiaoping Xu, Guangdong (CN); Mingxin Pan, Guangdong (CN)

(21) Appl. No.: 13/130,582

(22) PCT Filed: Apr. 16, 2010
(86) PCT No.: PCT/CN10/71828

§ 371 Date: May 23, 2011

(30) Foreign Application Priority Data
Mar. 19, 2010 (CN) 201010130360.5

(51) Int. Cl. C12N 5/02; C12M 1/12

(52) U.S. Cl. 435/383; 435/297.1

(57) ABSTRACT

A bioreactor includes a cylinder, a mandrel and a filter film. The cylinder includes two end walls and a circumferential wall interconnected with the both end walls; the two end walls and circumferential wall define together a reaction chamber. The mandrel is disposed between the two end walls. The mandrel has an inlet path and an outlet path provided on its both ends; the filter film wraps the mandrel to prevent passage of the first kind of substance while permit the passage of the second kind of substance; a gap is defined between the filter film and mandrel; at least one portion of the filter film is tied so as to divide the gap into multiple separate and isolated gap regions to prevent the second fluid from direct exiting from the outlet path through the gap. Also disclosed are control system and method for the bioreactor.

### BACKGROUND OF THE INVENTION

1. Field of the Invention

The invention relates to biomedical technology and more particularly, relates to a bioreactor, control system and control method used thereof.

2. The Related Art

Hepatic failure is symptom of the late period of many kinds of liver diseases during which health of the patient deteriorates heavily and death rate of the patients increases. Also, this symptom is difficult to expect. By now, liver transplantation is regarded as the most effective treatment. However, worldwide application and spread of this liver transplantation operation is seriously retarded due to insufficiency of donors and complication of operation. Treatment means such as bio-artificial liver device based on in vitro culture of liver cells is expected to provide an effective modern method for treatment of hepatic failure, just like artificial liver which had led to significant change in treatment of hepatic failure. Accordingly, it is strongly desired to design with efficiency a novel bioreactor to arrive at long term of in vitro culture of liver cells at large scale.

A bioreactor is the core component of a bio-artificial liver and performance thereof has direct effect on support characteristics of the artificial liver. The following paragraphs describe kinds of bioreactors which are under research or utilized currently and some bioreactors have been subject to clinical trials. However, none of these kinds of bioreactors can meet sufficiently clinical requirement.

One type of bioreactors is called hollow fiber bioreactor which is researched and utilized most frequently and widely. This type of reactor has the advantage of separating heterologous protein and at the same time, preventing damage to loading cells caused by pre-existing antibody related to heterologous cell antigen. This type of bioreactor is particularly suited to heterogous cell (such as pig liver cells). Currently this kind of reactor suffers from the following problems. Firstly, the volume is not large enough and cell loading amount is insufficient. The exchange area between nutrient solution and liver cells is restricted, thus having adverse effect on in vitro amplification reaction at large scale. Secondly, lateral holes of the translucent film are easy to be jammed by cell mass, hence generating adverse effect on exchange rate, long term and effective maintenance of function of the liver cells. As a result, this kind of bioreactor is not the best bio-artificial liver bioreactor.

Another type of bioreactor is known as flat-sheet bioreactor by which the liver cells directly grow on a flat sheet. This kind of bioreactor features its even distribution of liver cells and uniform microenvironment. However, the ratio of surface area to volume is decreased dramatically. In addition, reactor cells are cultured in a monolayer manner, and therefore the cells cannot survive for a long term and keep their function and activity. Moreover, these cells are unable to be amplified, thus failing to meet clinical requirement.

A further type of reactor is microcapsule suspension bioreactor in which the liver cells are wrapped with a sheet of translucent material to form porous microcapsules and then perfusion culture is performed. According to this kind of reactor, all the cells are in the same microenvironment and volume for cell culture is sufficiently enough, and occurrence risk of immune reaction is reduced. Unfortunately, energy exchange between materials inside and outside of the microcapsules is significantly limited due to existence of translucent film and aggregation of the liver cells. Additionally, research conducted by Hoshiba [11] further demonstrates that liver cells heavily reply on support walls. These cells will die if they are not attached to support materials. Consequently, the bioreactor of this kind is also not a best one for in vitro culture of liver cells at large scale.

A yet another type of bioreactor is named as perfusion bed/bracket bioreactor (agitation type) which is developed many years ago and has been employed extensively in both research and production. The cells together with bracket material are brought into suspension state by agitation. A sensor is provided on a top portion of a pot for consecutive monitoring of parameters of culture such as temperature, pH value, Po2, consumption of glucose and so on. The significant advantages of this kind of reactor are its ability of culturing many kinds of animal cells and easy enlargement of culture process. This kind of reactor however, has some disadvantages. For example, mechanical agitation is often accompanied with certain amount of shear which easily causes damage to the cells, thus further limiting its application.

Based on above analysis to kinds of prior art bioreactors, it is desired to make improvement on them.

Reference is made to U.S. Pat. No. 5,989,913 published on Nov. 23, 1999. What is disclosed is a culture vessel which includes:

a cylindrical vessel having a first and second end walls and a cylindrical wall defined therebetween, an inlet, an outlet, and first and second filters, the first and second filters having openings of a size that allows the passage of a fluid culture medium and cellular metabolic waste but prevents the passage of cells and cellular aggregates;

a culture chamber defined by the cylindrical wall, the first and second end walls, and the first and second filters, the culture chamber having an unobstructed longitudinal axis;

means for rotating the cylindrical vessel about, a horizontal longitudinal axis; and

a pump for maintaining a flow of fluid culture medium through the culture chamber.

After tens of years of research and design by NASA, a rotary cell culture system (RCCS) for use in microgravity life science field has presently successfully and extensively applied to a variety of engineering fields such as keratocytes of rabbits, skeletal muscle cells, osteoblasts and the like. The newest one of the series of products of NASA, that is, rotary cell perfusion microgravity culture system (RCMW), has the same construction with that disclosed in U.S. Pat. No. 5,989,913. By the above same construction, micro-carriers and cells inside the vessel can be placed in a suspension state by rotating horizontally the culture vessel. Moreover, bidirectional circulation of oxygen, nutrients and metabolite inside the vessel is realized by using a peristaltic pump disposed outside of the vessel. However, this kind of bioreactor still has some problems found during operation of it at early period. For example, supply of nutrient is insufficient, perfusion is not uniform, and the vessel is often jammed.

At first, substance exchange efficiency of the culture vessel is low. It is because the longitudinal axis of the vessel is wrapped by the filter films at its inlet and outlet, which leads to passage of part of culture medium through the filter film and exchange of nutrient and oxygen with culture medium of another culture chamber outside of the film, thus realizing “effective exchange”. However, part of culture medium flows directly out of the culture vessel across a passage defined by a gap between the filter film and longitudinal axis, thus failing to exchange nutrient and oxygen, and leading to nutrient insufficiency of the cell tissues inside the culture vessel and finally becoming “invalid circulation”.

Secondly, perfusion performed inside the culture vessel is not uniform and dead space exists. In RCMW cycle mode, outer cycle of the filter film may be enhanced by improving permeability of the filter film and accordingly, reducing invalid circulation. However, as the fluid pressure at the middle portion of the culture vessel (close to the rotation axis) is lower than the ambient fluid pressure, the flow speed and change rate of the culture medium at middle portion of the culture vessel are higher, while those of other portion of the culture vessel are lower, resulting in uneven perfusion inside the vessel and generation of dead space outside the culture vessel.

Furthermore, in RCMW cycle mode, cells and micro-carriers will be jammed at the outlets due to flow of the fluid inside the culture vessel is only toward one direction and the total area of the outlets of culture fluid is too small and too crowed (four side holes).

### SUMMARY OF THE INVENTION

One primary object of the invention is to provide a bioreactor capable of eliminating perfusion dead space.

Another object of the invention is to provide a bioreactor control system which can enhance the exchange efficiency and uniformity of two kinds of fluid engaged in reaction and overcome dead space and jamming problems resulted during exchange period.

A further object of the invention is to provide a bioreactor control method pertaining to the aforementioned control system.

To obtain the above objects, a bioreactor is provided including a cylinder, a mandrel and a filter film. The cylinder comprises two end walls and a circumferential wall interconnected with the both end walls; the two end walls and circumferential wall define together a reaction chamber into which reaction happens between a first fluid containing a first kind of substance and a second fluid containing a second kind of substance; the mandrel is disposed between and traverse the two end walls; the mandrel has an inlet path through which the second fluid can enter the reaction chamber and an outlet path through which the second fluid flows out of the reaction chamber provided on its both ends; the filter film wraps the mandrel to prevent passage of the first kind of substance while permit the passage of the second kind of substance; a gap is defined between the filter film and mandrel; at least one portion of the filter film is tied so as to divide the gap into multiple separate and isolated gap regions to prevent the second fluid from direct exiting from the outlet path through the gap.

A bioreactor control system, includes a bioreactor; a storage bottle for holding the second fluid containing the second kind of substance; a dynamic pump for ensuring that the second fluid inside the storage bottle passes through the reaction chamber of the reactor and then returns to the storage bottle so as to form a cycle circuit; and a motor for driving the bioreactor rotated around its mandrel.

A control method for a bioreactor includes the following steps: filling in advance the reaction chamber of the bioreactor with a first fluid containing a first kind of substance; preparing a second fluid containing a second kind of substance; providing power such that, the second fluid enters the reaction chamber via the inlet path of the reactor, reacts with the first fluid in the reaction chamber, and then reflows via the outlet of the reactor, thus forming a cycle circuit; providing power such that the reactor rotates around its mandrel, thus making the first and second fluids inside the reaction chamber reacting more uniformly.

Phenol red experiments have been made using the control system and method of the invention and, better effects have been observed.

Summarily, the bioreactor, control system and method thereof provided by the invention are specifically suitable for application in bio-artificial liver. Problems such as perfusion ununiformity, existence of dead space, blockage and low exchange rate existing in prior art technology are completely overcome by the invention. In other words, a better auxiliary device is provided by the invention.

### BRIEF DESCRIPTION OF THE DRAWINGS

The present invention will be apparent to those skilled in the art by reading the following description of a preferred embodiment thereof, with reference to the accompanying drawings, in which:

FIGS. 1 and 2 are longitudinal cross-sectional views of a bioreactor according to different embodiments of the invention, illustrating interior construction thereof;

FIG. 3 is a variation of the bioreactor of FIG. 2, showing specifically an additional mesh barrel;

FIG. 4 shows an enlarged view of portion A of FIG. 3;

FIGS. 5a, 5b, 5c and 5d are views showing respectively mesh barrels of FIG. 3 in expanded states, illustrating meshes of different shapes;

FIG. 6 shows a longitudinal cross-sectional view of a bioreactor according to an embodiment of the invention, illustrating interior construction thereof;

FIG. 7 is an enlarged view of portion B of FIG. 6;

FIG. 8 shows a schematic view of a bioreactor control system provided with two kinds of oxygenators according to an embodiment of the invention;

FIG. 9 shows a schematic view of a bioreactor control system provided with only one oxygenator of FIG. 8 according to an embodiment of the invention;

FIG. 10 shows a schematic view of a bioreactor control system provided with an oxygenator different from that of FIG. 8 according to an embodiment of the invention;

FIG. 11 shows a schematic view of a bioreactor control system according to an embodiment of the invention, the control system being different from that of FIG. 10 in flow direction of a second fluid.

FIG. 12 shows a longitudinal cross-sectional view of an oxygenator according to an embodiment of the invention, denoting interior structure thereof; and

FIG. 13 shows schematically interior construction of a direction controller according to an embodiment of the invention.

### DETAILED DESCRIPTION OF THE PREFERRED EMBODIMENT

Now various embodiments of the invention will be described in great detail in conjunction with the accompanying drawings.

According to the invention, biochemical reaction may take place between a first fluid and a second fluid. For two kinds of fluid between which biochemical reaction may happen, the biochemical reaction will make one of them to be target which has been ready for medicine preparation or treatment purposes. The biochemical reaction happens actually between a first kind of substance dissolved or contained in the first fluid and a second kind of substance dissolved or contained in the second fluid. Take an example, during period of cell culture simulating growing process of a bio-artificial liver, a culture medium, which contains cells to be cultured and functions as a first fluid, is perfused into a bioreactor. The first kind of substance is cells. Then, another culture medium, which contains nutrients (such as amino acid and glucose) and oxygen and serves as a second fluid, passes the bioreactor so as to culture the cells inside the reactor. The nutrients and oxygen described above constitute the second kind of substance. Take another example, during the period of treatment simulating bio-artificial liver; the first fluid perfused into the bioreactor is healthy human blood containing healthy cells which are the first kind of substance aforementioned. The second fluid passing through the bioreactor is the blood of a patient. Metabolic waste and toxin inside the patient blood become the second kind of substance in this instance. When contacting the first fluid, the metabolic waste and toxin are all ingested by the healthy cells contained in blood. The second fluid flowing out of the bioreactor will become relatively healthy blood. The above two examples explain two kinds of biochemical reactions happened inside the bioreactor and based on cell mechanism. Similarly, person of ordinary skill in the art will certainly know that the bioreactor provided by the invention may also find their application in other kinds of biochemical reactions.

It is clear from above two examples that the first and second fluids of the invention share the same ingredients such as the culture medium. Furthermore, the ingredients of the second fluid will be changed when passing through the bioreactor. More particularly, the amount of the second kind of substance will be reduced or even eliminated completely due to occurrence of biochemical reaction between the second kind of substance (for example, the nutrients and/or oxygen) and first kind of substance (such as cells) inside the reaction chamber. Also, the common portion such as the culture medium may have been exchanged between the first and second fluids. When the second fluid is supplied initially, the second kind of substance thereof only contains some nutrients. When oxygen is infused into the second fluid, the second kind of substance thereof will contain the nutrients and oxygen. Part of the second kind of substance is significantly reduced or even eliminated when the second fluid flows out of the bioreactor. Apparently, change of ingredients, as a dynamic concept, will not have influence on understanding of the “fluid” of the invention.

The first example described above will be taken to explain the principle of the invention. In the first example, culture medium containing the cells to be cultured is taken as the first fluid, while another culture medium including the nutrients and oxygen is used as the second fluid. The reaction chamber of the bioreactor is also named as culture chamber for easy understanding of the term by ordinary person of related art.

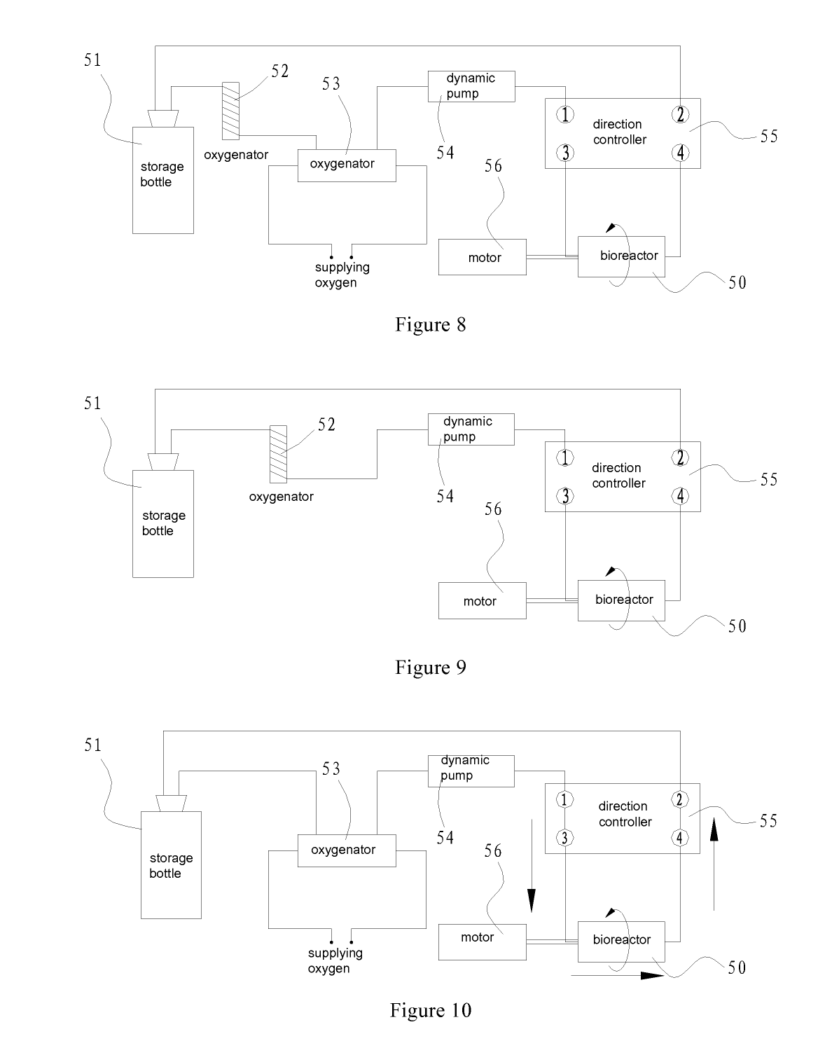

Reference is made to FIGS. 8-11 which denote construction of the bioreactor control system of the invention. As shown in figures, the bioreactor control system includes a bioreactor 50, a motor 56, a flow direction controller 55, a dynamic pump 54, a pair of oxygenators 52, 53, and a storage bottle 51, all of which define together a cycle circuit. More detailed description to the above components of the bioreactor of the invention will be provided below.

The bioreactor 50 provided by the invention may take many forms one of which is illustrated in FIG. 1. Referring to FIG. 1 and according to a first embodiment of the invention, the bioreactor 50 is cylinder-shaped and includes a cylinder 1, a mandrel 3 and a filter film 2.

The cylinder 1 has a pair of end walls 11, 12 and a circumferential wall 13 interconnected with both of the end walls 11, 12. The pair of end walls 11, 13 and circumferential wall 13 define together a reaction chamber 10 in which biochemical reaction may happen between a culture medium (the first fluid) containing cells and another culture medium (the second fluid) containing nutrients and oxygen.

The mandrel 3 is disposed and extended between the two end walls 11 and 12. The mandrel 3 is substantially solid except for its inlet path 31 and outlet path 32. Preferably, the axis of the mandrel 3 coincides with that of the cylinder 1. The mandrel 3 has an inlet path 31 at one side thereof by which the culture medium (the second fluid) containing nutrients and oxygen may enter the reaction chamber 10 and an outlet path 32 at the other side thereof by which the culture medium (the second fluid) after reaction may flow out of the reaction chamber 10. The inlet path 31 is a hole which is defined at the center of the first end wall 11, extended axially into the mandrel 3 and then radially extended out of the mandrel 3 so as to communicate with the reaction chamber 10. Corresponding to the inlet path 31, an outer inlet 310 is provided on the first end wall 11 at an outer side thereof for introducing the culture medium containing the nutrients and oxygen therein. The mandrel 3 has one or more inner inlets 313 defined in its circumferential surface at one side thereof By the same token, the outlet path 32 is also a hole which is defined at the center of the second end wall 12, extended inside the mandrel 3 axially and then extended radially out of the mandrel 3 so as to communicate with the reaction chamber 10. Corresponding to the outlet path 32, the second end wall 12 has an outer outlet 320 defined at the outside thereof for making the culture medium flowing back to the storage bottle 51 after reaction. The mandrel 3 has one or more inner outlets 323 defined in its circumferential surface at an opposite side thereof through which the culture medium after reaction may flow out of the reaction chamber 10. Obviously in most instances, the location and dimension of the inner inlets 313 of the inlet path 31 and inner outlets 323 of the outlet path 32 determine the movement distance of the fluids inside the reaction chamber 10.

The filter film 2 is wrapped on the circumferential surface of the mandrel 3 and therefore, takes shape of barrel. Plural small holes of suitable diameter (not shown) are defined in the filter film 2 for preventing passage of the first fluid especially the first kind of substance, while permitting passage of the second fluid especially the second kind of substance. More specifically, as the diameter of cells is larger than that of the nutrients and oxygen, the diameter of the small holes of the filter film 2 is designed to be larger than the second substance and smaller than the first substance, thus realizing the above function. Gap 20 may be easily formed between the filter film 2 and mandrel 3 due to softness and flexibility of the filter film 2. As a result, when passing through the inlet path 31, part of the second fluid will come into the reaction chamber 10 through the filter film 2, while the other part thereof will reach the inner outlet 323 of the outlet path 32 through the gap 20 and then directly flows out of the reaction chamber 10 via the outlet path 32. To prevent this, a tying member 400 is provided to tie the barrel of the filter film 2 at its central portion along longitudinal direction, as shown in FIG. 1. As such, the filter film 2 is pressed tightly against the mandrel 3 at a location where the filter film 2 is tied and as a result, the gap 20 is divided into two separate and isolated gap regions 201 and 203. Because the two gap regions 201 and 203 are not communicated with each other, the second fluid will completely enters the reaction chamber 10 to take part in the reaction. Consequently, exchange rate between the first and second fluids will be enhanced.

The tying member 400 is designed to have a circular shape and no specific dimension may be required as to its radial width. The tying member 400 may have a circular cross-section. The tying member 400 may be suitably resilient in order to make adjustment to tying location by the user. Of course, it is also possible for the tying member 400 to extend along radial direction so as not to block the circulation of the fluids inside the reaction chamber 10. In a typical application, a rubber ring made of rubber material may be utilized directly as the tying member 400. In addition, in some examples not shown in figures, tying location of the tying member 400 relative to the filter film 2 may be adjusted. For example, the tying member 400 may be located either at the inlet path 31 or at the outlet path 32 for purposes of prevention of “invalid circulation”.

It is noted from FIG. 1 that dead spaces, into which fluids may be contained, may be easily generated between the filter film 2 and the mandrel 3 at location between the inner inlet 313 of the inlet path 31 and the first end wall 11, or at location between the inner outlet 323 of the outlet path 32 and the second end wall 12. To bar generation of the dead spaces, tying members 401 and 402 may be provided at these locations. Alternatively, the inner inlet 313 of the inlet path 31 and inner outlet 323 of the outlet path 32 may be designed to be close to the end walls 11 and 12 respectively, thus rendering tying unnecessary.

Note that the tying members 401 and 402 disposed at both sides of the filter film 2 are different from the tying member 400 placed at the central portion of the film 2. The tying members 401 and 402 are located beyond the flowing movement path of the fluid inside the reaction chamber 10 for preventing generation of dead spaces between the filter film 2 and mandrel 3, while the tying member 400 is located within the flowing movement path for preventing escape of the second fluid from the gap 20 between the film 2 and mandrel 3. Theoretically, in some circumstances, it may be unnecessary to provide the tying member 401 if the distance between the inner inlet 313 of the inlet path 31 and the first end wall 11 is short enough and the film 2 and mandrel 3 are sealed together by the first end wall 11. By the same reason, the tying member 402 may also be eliminated if the distance between the inner outlet 323 of the outlet path 32 and second end wall 12 is small sufficiently and the film 2 and mandrel 3 are sealed together by the second end wall 12.

In addition, to facilitate supplying sample material to the reaction chamber 10 and taking the same from the chamber 10, a sample taking opening 14 and a sample supplying opening 15 may be formed on the circumferential wall 13 of the cylinder 1. When not in use, these openings 14 and 15 are covered with plugs 140 and 150 respectively and when in use, the plugs are removed from these openings.

A control system and method adapted to the bioreactor of the embodiment will be illustrated below with great detail to demonstrate the good effects of the invention.

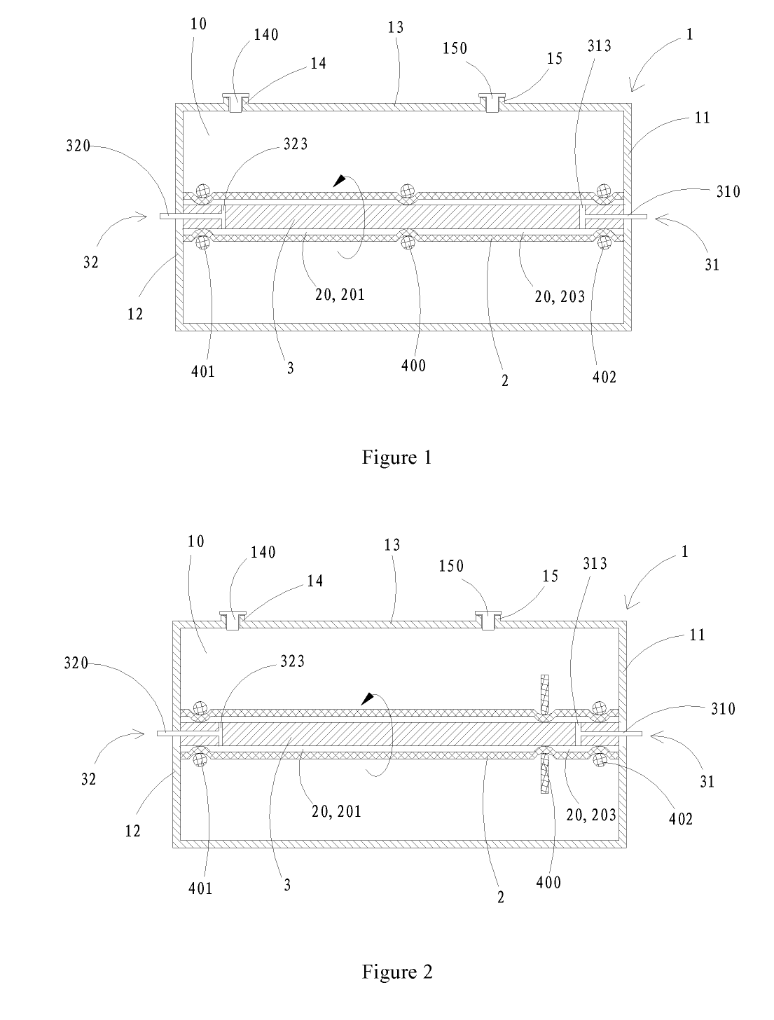

With reference to FIG. 2 and according to another embodiment of the invention, the bioreactor has the similar structure with the aforementioned reactor. In this embodiment, the reactor is of a cylinder shape and includes a cylinder 1, a mandrel 3 and a filter film 2.

The cylinder 1 has two end walls 11 and 12 and a circumferential wall 14 connected with both of the end walls 11 and 12. A reaction chamber 10 is defined by the two end walls 11, 12 and circumferential wall 13 for performing biochemical reaction between a culture medium (the first fluid) including cells and another culture medium (the second fluid) including nutrients and oxygen.

The mandrel 3 is disposed and extended between the two end walls 11 and 12. The mandrel 3 is substantially solid except for its inlet path 31 and outlet path 32. Preferably, the axis of the mandrel 3 coincides with that of the cylinder 1. The mandrel 3 has an inlet path 31 at one side thereof by which the culture medium (the second fluid) containing nutrients and oxygen may enter the reaction chamber 10 and an outlet path 32 at the other side thereof by which the culture medium (the second fluid) after reaction may flow out of the reaction chamber 10. The inlet path 31 is a hole which is defined at the center of the first end wall 11, extended axially into the mandrel 3 and then radially extended out of the mandrel 3 so as to communicate with the reaction chamber 10. Corresponding to the inlet path 31, an outer inlet 310 is provided on the first end wall 11 at an outer side thereof for introducing the culture medium containing the nutrients and oxygen therein. The mandrel 3 has one or more inner inlets 313 defined in its circumferential surface at one side thereof By the same token, the outlet path 32 is also a hole which is defined at the center of the second end wall 12, extended inside the mandrel 3 axially and then extended radially out of the mandrel 3 so as to communicate with the reaction chamber 10. Corresponding to the outlet path 32, the second end wall 12 has an outer outlet 320 defined at the outside thereof for making the culture medium flowing back to the storage bottle 51 after reaction. The mandrel 3 has one or more inner outlets 323 defined in its circumferential surface at an opposite side thereof through which the culture medium after reaction may flow out of the reaction chamber 10. Obviously in most instances, the location and dimension of the inner inlets 313 of the inlet path 31 and inner outlets 323 of the outlet path 32 determine the movement distance of the fluids inside the reaction chamber 10.

The filter film 2 is wrapped on the circumferential surface of the mandrel 3 and therefore, takes shape of barrel. Plural small holes of suitable diameter (not shown) are defined in the filter film 2 for preventing passage of the first fluid especially the first kind of substance, while permitting passage of the second fluid especially the second kind of substance. More specifically, as the diameter of cells is larger than that of the nutrients and oxygen, the diameter of the small holes of the filter film 2 is designed to be larger than the second substance and smaller than the first substance, thus realizing the above function. Gap 20 may be easily formed between the filter film 2 and mandrel 3 due to softness and flexibility of the filter film 2. As a result, when passing through the inlet path 31, part of the second fluid will come into the reaction chamber 10 through the filter film 2, while the other part thereof will reach the inner outlet 323 of the outlet path 32 through the gap 20 and then directly flows out of the reaction chamber 10 via the outlet path 32. To prevent this, a tying member 400 is provided to tie the barrel of the filter film 2 at its central portion along longitudinal direction, as shown in FIG. 1. As such, the filter film 2 is pressed tightly against the mandrel 3 at a location where the filter film 2 is tied and as a result, the gap 20 is divided into two separate and isolated gap regions 201 and 203. Because the two gap regions 201 and 203 are not communicated with each other, the second fluid will completely enters the reaction chamber 10 to take part in the reaction. Consequently, exchange rate between the first and second fluids will be enhanced.

The tying member 400 is designed to have a pie shape. In other words, the member 400 has a certain width along its diameter direction. The radius of the member 400 may be larger or smaller than that of the cylinder 1. For example, assuming the radius of the cylinder 1 is R, the radius r of the member 400 may be within 0.3R-0.7R. Preferably, r=R/2. In this example, the member 400 needs to be made of stiff material such as hard metal, wood board, plastic, ceramic and so on in order to have sufficient resistance to impact caused by fluid and resistance to deformation. Preferably, the member 400 is made of metal. The member 400 of certain hardness will make it easy for the second fluid entered the reaction chamber 10 to flow in a radiation pattern, thus rendering the exchange between the mixed fluids inside the reaction chamber 10 more even.

The tying member 400 is designed to have a circular cross section. An axle hole (not shown) is defined in the center of the tying member 400 through which the mandrel 3 together with the filter film 2 may pass. Moreover, the axle hole is configured to make the filter film 2 be pressed against the mandrel 3 by the tying member 400. Compared with the above embodiment, this embodiment gets the tying member 400 closer to the inner inlet 313 of the inlet path 31. When the bioreactor 50 of this embodiment is applied to corresponding control system, it may be obtained more beneficial effects.

It is noted from FIG. 2 that dead spaces, into which fluids may be contained, may also be easily generated between the filter film 2 and the mandrel 3 at location between the inner inlet 313 of the inlet path 31 and the first end wall 11, or at location between the inner outlet 323 of the outlet path 32 and the second end wall 12. To prevent generation of the dead spaces, tying members 401 and 402 having small cross section may be provided at these locations. Alternatively, the inner inlet 313 of the inlet path 31 and inner outlet 323 of the outlet path 32 may be designed to be close to the end walls 11 and 12 respectively, thus rendering tying unnecessary.

Note that the tying members 401 and 402 disposed at both sides of the filter film 2 are different from the tying member 400 placed near the inner inlet 313. The tying members 401 and 402 are located beyond the flowing movement path of the fluid inside the reaction chamber 10 for preventing generation of dead spaces between the filter film 2 and mandrel 3, while the tying member 400 is located within the flowing movement path for preventing escape of the second fluid from the gap 20 defined between the film 2 and mandrel 3. In addition, the tying member 400 has another function of leading to flow of radiation pattern for the second fluid at the circumference of the tying member 400 when it enters from the inner inlet 313, hence diffusing the second fluid uniformly into the reaction chamber 10. Accordingly, exchange between the first and second fluids is significantly uniform everywhere inside the reaction chamber 10.

Theoretically, in some circumstances, it may be unnecessary to provide the tying member 401 if the distance between the inner inlet 313 of the inlet path 31 and the first end wall 11 is short enough and the film 2 and mandrel 3 are sealed together by the first end wall 11. By the same reason, the tying member 402 may also be eliminated if the distance between the inner outlet 323 of the outlet path 32 and second end wall 12 is small sufficiently and the film 2 and mandrel 3 are sealed together by the second end wall 12.

In addition, to facilitate supplying sample material to the reaction chamber 10 and taking the same from the chamber 10, a sample taking opening 14 and a sample supplying opening 15 may be formed on the circumferential wall 13 of the cylinder 1. When not in use, these openings 14 and 15 are covered with plugs 140 and 150 respectively and when in use, the plugs are removed from these openings.

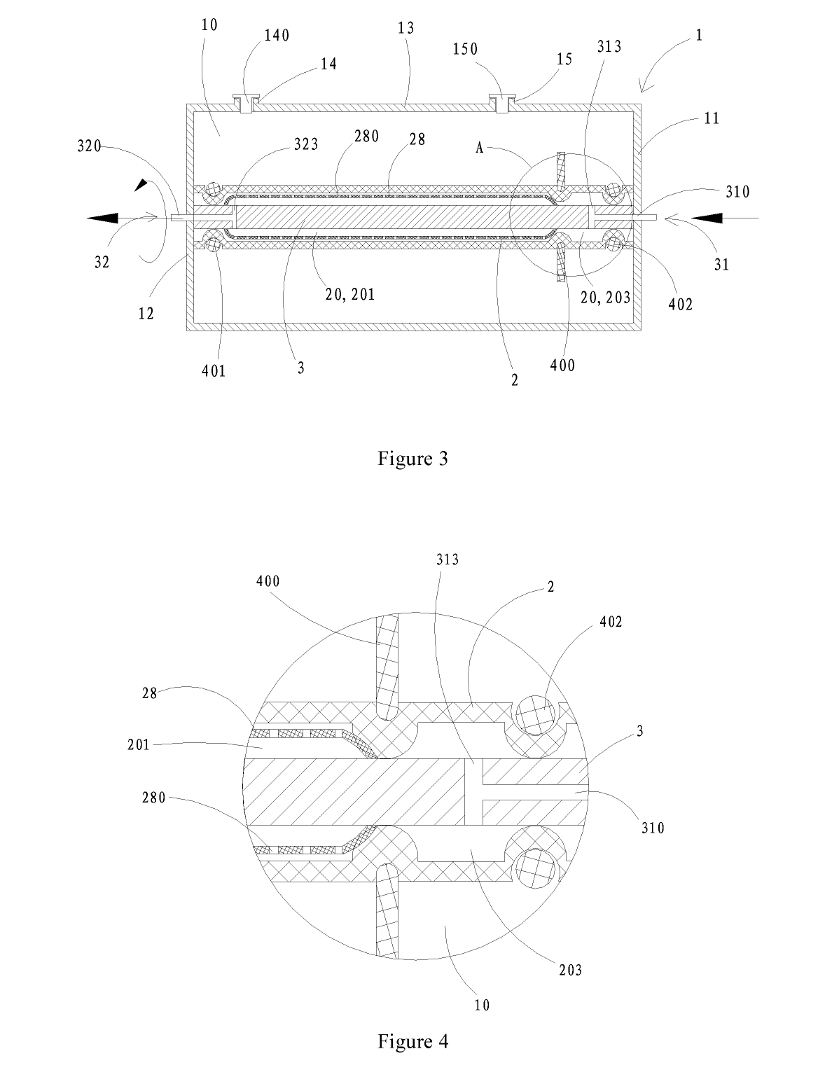

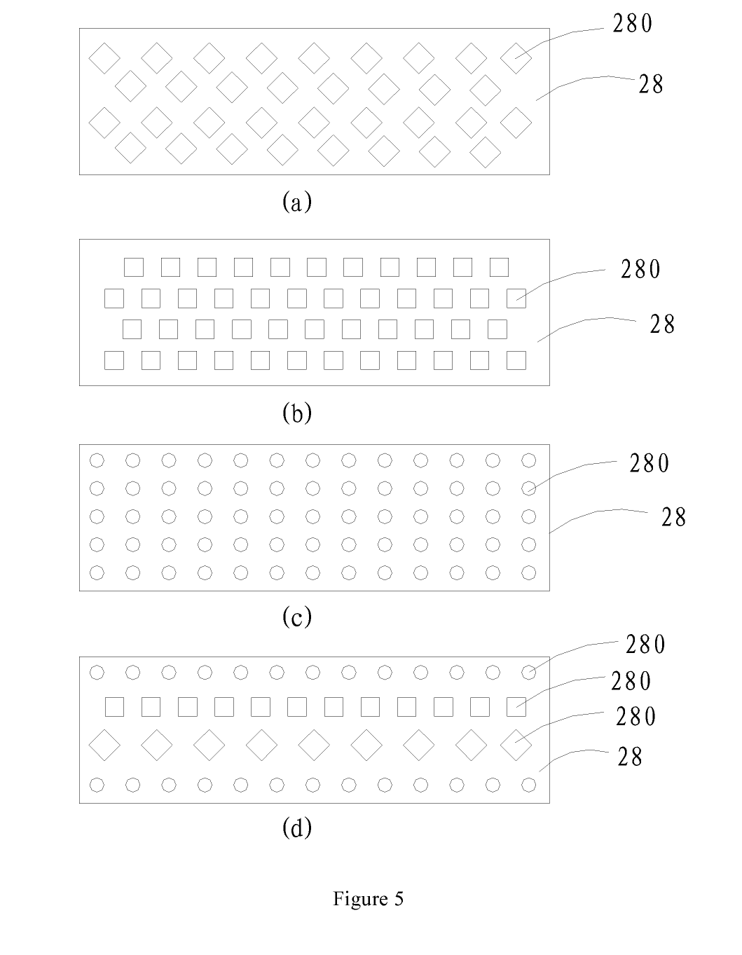

An improvement may be made to the bioreactor of the embodiment. Reference is made to FIGS. 3 and 4. FIG. 4 shows an enlarged view of portion A of FIG. 3. As shown in FIG. 3, a gap 20 is defined by the filter film 2 and mandrel 3 and the gap 20 is further divided by a tying member 400 into two gap regions 201 and 203. A mesh barrel 28 is disposed in the gap region 201 occupied by the outlet path 32 especially the inner outlet 323 thereof. The mesh barrel 28 has a barrel shape corresponding to the mandrel 3. A plurality of meshes 280 is defined in the circumferential wall 13 thereof. The shape of the meshes 280 maybe designed freely. As shown in FIG. 5a-5d, the arrangement of the meshes 280 may be regular or irregular. The meshes 280 may take any form such as rectangular, square (FIG. 5a), diamond (FIG. 5b), circle (FIG. 5c), triangle and any combination of the above shapes (FIG. 5d). The arrangement of the meshes 280 is helpful. For example, after reaction, the second fluid comes across the filter film 2 and then enters the gap region 201. After that, the second fluid passes through the mesh barrel 28 before it get into the outlet path 32 via the inner outlet 323. After passing through the filter film 2, the second fluid enters the outlet path 32 via the plurality of meshes 280. As a result, it seems as though a number of “inlets” was formed in the surface of the filter film 2. Therefore, mixture of fluids inside the reaction chamber 10 will not accumulate at any site. Rather, the mixture of the fluids will flow from the entire surface of the film 2 to the gap region 201, pass through the mesh barrel 28 and finally enter the outlet path 32. As such, the cells inside the reaction chamber 10 will not accumulate on the inner outlet 323 of the outlet path 32 at a location corresponding to the film 2.

It can be understood from the description of this embodiment of bioreactor that the tying member 400 of this embodiment is different from the aforementioned embodiments in size, location and addition of a mesh barrel 28. It can also be understood that the mesh barrel 28 of this embodiment may also be applied to the other embodiment of the invention. In this situation, the mesh barrel 28 may be equipped into the gap region 201 corresponding to the outlet path 32, as long as there is a fluid outlet for avoiding block of the cells inside the reaction chamber 10.

A control system and method adapted to the bioreactor of the embodiment will be illustrated below with great detail to demonstrate the good effects of the invention.

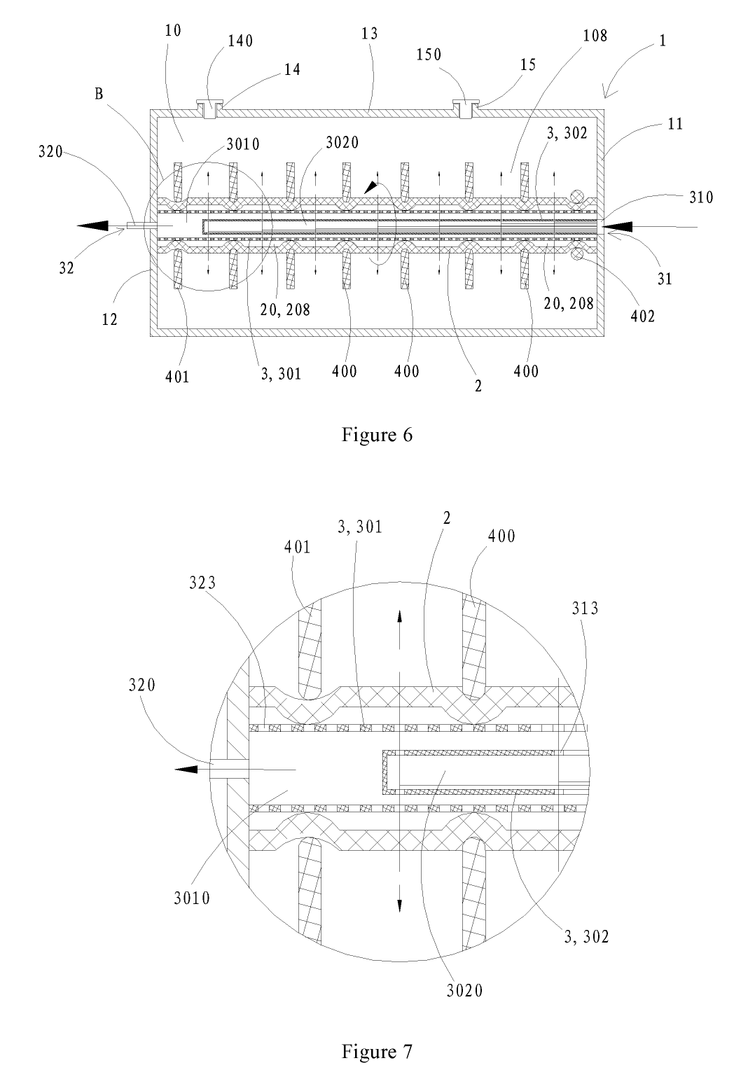

Reference is made to FIGS. 6 and 7 which show another embodiment of the bioreactor of the invention. FIG. 7 shows an enlarged view of portion B of FIG. 6. The bioreactor of this embodiment is improved upon the above embodiments and has the similar construction.

In this embodiment, the reactor is of a cylinder shape and includes a cylinder 1, a mandrel 3 and a filter film 2.

The cylinder 1 has two end walls 11 and 12 and a circumferential wall 14 connected with both of the end walls 11 and 12. A reaction chamber 10 is defined by the two end walls 11, 12 and circumferential wall 13 for performing biochemical reaction between a culture medium (the first fluid) including cells and another culture medium (the second fluid) including nutrients and oxygen.

The mandrel 3 is disposed and extended between the two end walls 11 and 12. The mandrel 3 is substantially solid except for its inlet path 31 and outlet path 32. Preferably, the axis of the mandrel 3 coincides with that of the cylinder 1. The mandrel 3 has an inner barrel 302 and an outer barrel 301. The outer barrel 301 is hollowed and has a plurality of through holes defined in its barrel wall along an axial direction to form the inner outlet 323. In addition, one end of the outer barrel 301 extends beyond the second end wall 12 so as to form the outer outlet 320. As such, an entire outlet path 32 for the outer barrel 301 is defined by the inner outlet 323, the hollowed portion 3010, and the outer outlet 320. The inner barrel 302 is mounted inside the mandrel 3 and has a diameter smaller than that of the outer barrel 301. The inner barrel 302 also has a hollowed portion 3020 one end of which adjacent to the second end wall 12 is sealed, while the other end adjacent to the first end wall 11 is open. A plurality of through holes is defined in the barrel wall of the inner barrel 302 along an axial direction so as to form several inner inlets 313. The open end of the inner barrel 302 is connected with the first end wall 11 in order to form an outer inlet 310 beyond the first end wall 11. Accordingly, an inlet path 31 is defined by the outer inlet 310, the hollowed portion 3020, and the inner inlet 313. Resultantly, both ends of the mandrel 3 form an inlet path 31 through which a culture medium (the second fluid) containing nutrients and oxygen may enter the reaction chamber 10, and an outlet path 32 from which another culture medium (the first fluid) after reaction may exit out of the chamber 10. Preferably, the length of the inner barrel 302 is no less than half length of the outer barrel 301. By this way, the inlet path 31 will extend a long distance, The second fluid at the inlet path 31 is permitted to flow into the reaction chamber 10 from a relatively wide transverse location with a gradually slowed speed. Similarly, as the outer barrel 301 runs across the entire axial length of the chamber 10, the second fluid may also uniformly flow into the outlet path 32 from the entire barrel wall thereof. Understandingly, the first kind of substance inside the chamber 10 will not accumulate at any site due to arrangement of the plural outlets or inlets.

Clearly, on one hand, as the outer barrel 301 is hollowed, the second fluid passed through the outer inlet 310 of the inner barrel 302 comes in the reaction chamber 10 via the outlet path 32 especially the hollowed portion 3010 of the outer barrel 301. On the other hand, the second fluid after reaction enters the outlet path 32 through the inner outlet 323 of the outer barrel 301. Actually, the through holes defined in the surface of the outer barrel 301 permits flowing of the second fluid to which reaction has not yet happened into the chamber 10, and also permits flow of the second fluid, to which reaction has occurred, into the outlet path 32.

The filter film 2 is wrapped on the circumferential surface of the mandrel 3 and therefore, takes shape of barrel. Plural small holes of suitable diameter (not shown) are defined in the filter film 2 for preventing passage of the first fluid especially the first kind of substance, while permitting passage of the second fluid especially the second kind of substance. More specifically, as the diameter of cells is larger than that of the nutrients and oxygen, the diameter of the small holes of the filter film 2 is designed to be larger than the second substance and smaller than the first substance, thus realizing the above function. Gap 20 may be easily formed between the filter film 2 and mandrel 3 due to softness and flexibility of the filter film 2. As a result, when passing through the inlet path 31, part of the second fluid will come into the reaction chamber 10 through the filter film 2, while the other part thereof will reach the inner outlet 323 of the outlet path 32 through the gap 20 and then directly flows out of the reaction chamber 10 via the outlet path 32. To prevent this, a plurality of tying members 400 is provided and distributed uniformly to tie the barrel of the filter film 2 at its central portion along longitudinal direction, as shown in FIG. 6. As such, the filter film 2 is pressed tightly against the mandrel 3 at a location where the filter film 2 is tied and as a result, the gap 20 is divided into plural separate and isolated gap regions 208. Because the gap regions 208 are not communicated with each other, the second fluid will completely enters the reaction chamber 10 to take part in the reaction and then flows out of the chamber 10. Consequently, exchange rate between the first and second fluids will be enhanced.

The tying member 400 is designed to have a circular cross section. An axle hole (not shown) is defined in the center of the tying member 400 through which the mandrel 3 together with the filter film 2 may pass. The tying member 400 is designed to have a pie shape. In other words, the member 400 has a certain width along its diameter direction. The radius of the member 400 may be larger or smaller than that of the cylinder 1. For example, assuming the radius of the cylinder 1 is R, the radius r of the member 400 may be within 0.3R-0.7R. Preferably, r=R/2. In this example, the member 400 needs to be made of stiff material such as hard metal, wood board, plastic, ceramic and so on in order to have sufficient resistance to impact caused by fluid and resistance to deformation. Preferably, the member 400 is made of metal.

Arrangement of multiple tying members 400 of large surface area leads to division of the reaction chamber 10 into multiple reaction regions 108 of short cylinder type. All these reaction regions 108 are communicated with each other at their periphery and have their individual inner inlets 313 and inner outlets 323 respectively. As a result, each reaction region 108 defines a small reaction chamber. As these reaction regions 108 are separate from each other and relatively small, the second fluid which entered through the outlet path 32 can get in each reaction region 108 with a gradually slowed speed and then perform biochemical reaction with the first fluid inside each reaction region 108. After completion of biochemical reaction, the second fluid contained in each reaction region 108 may directly enter the outlet path 32 through corresponding inner outlet 323. As the large reaction chamber 10 is subdivided into multiple small reaction regions, the biochemical reaction may be performed more evenly.

With reference to FIG. 6, rubber rings with small cross-section area such as tying members 402 may be used at locations adjacent the two end walls 11 and 12 so as to enhance the tight contact between the filter film 2 and the outer barrel 301 of the mandrel 3. The tying member used in this situation may be designed to have a large surface area such as that represented by numeral 401.

In addition, to facilitate supplying sample material to the reaction chamber 10 and taking the same from the chamber 10, a sample taking opening 14 and a sample supplying opening 15 may be formed on the circumferential wall 13 of the cylinder 1. When not in use, these openings 14 and 15 are covered with plugs 140 and 150 respectively and when in use, the plugs are removed from these openings.

The two embodiments of the bioreactor described above each have a mesh barrel 28. In this embodiment however, no mesh barrel 28 is utilized, it is because multiple through holes defined in the outer barrel 301 of the mandrel 3 along axial direction have the same function as the mesh barrel 28.

It is clear from the above embodiment that the reaction chamber 10 is divided into plural small reaction regions which makes it different from the other embodiments described hereinbefore. A control system and method adapted to the bioreactor of the embodiment will be illustrated below with great detail to demonstrate the good effects of the invention.

Three embodiments of the bioreactor of the invention have been discussed above and now, other components of the bioreactor control system of the invention will be discussed below.

With reference to FIG. 8-11, according to the invention, a motor 56 is mainly used to drive the bioreactor 50 to rotate around its axis. As the mandrel 3 and cylinder 1 of the reactor 50 share the identical axis, rotation of the mandrel around its own axis will make the whole cylinder 1 rotate, thus resulting in rotation of the entire bioreactor 50. The rotation may be unidirectional or bidirectional. The rotation direction of the motor 56 has no influence on the implementation of the invention.

The storage bottle 51 of the invention serves to retain the culture medium including the nutrients.

In the bioreactor control system of the invention, the storage bottle 51 is communicated with both the outer inlet 310 and outer outlet 320 of the reactor 50 through conduits, thereby forming a cycle circuit. A dynamic pump 54 is provided for driving the second fluid in the storage bottle 51 to be circulated in the cycle circuit. To keep the oxygen inside the culture medium inside the storage bottle 51, at least one oxygenator 52 is needed which provides oxygen originated from nature air or other dedicated source (not shown) to the second fluid. In addition, a direction controller 55 may be equipped for the cycle circuit.

The direction controller 55 of the invention includes multiple three-way valves (not shown). The controller 55 has two input ends and two output ends. The direction change of the two input ends and output ends of the controller 55 is obtained by electronic or manual manner. The direction change is made by changing the connection among the three-way valves.

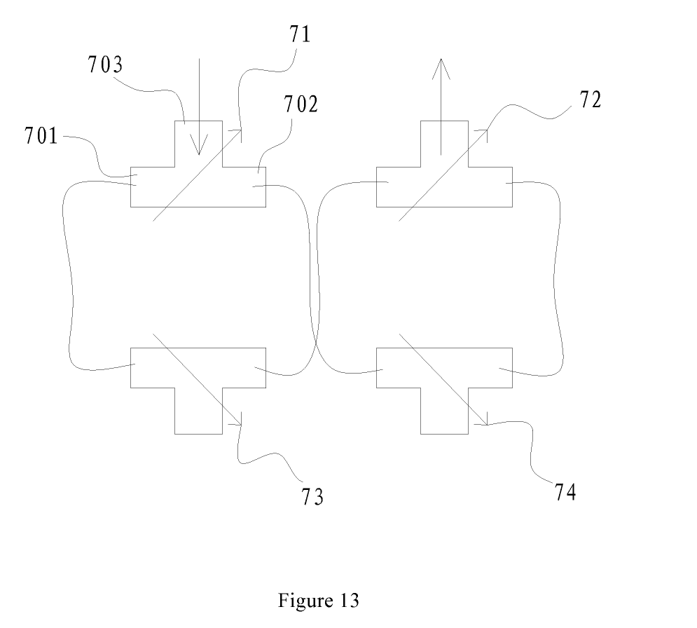

Referring to FIG. 13, the direction controller 55 includes two fixed electrically controlled three-way valves 71, 72, and two direction-changeable electrically controlled three-way valves 73, 74. Each three-way valve has two fluid-passing ports 701 and 702, and a vertical port 703. The two fluid-passing ports 701 and 702 of the first fixed electrically controlled three-way valve 71 are communicated with one fluid-passing port of each direction-changeable electrically controlled three-way valve 73 and 74. The vertical port 703 of the first valve 71 may be communicated with the dynamic pump 54. The two fluid-passing ports of the second fixed electrically controlled three-way valve 72 are also communicated with one fluid-passing port of each direction-changeable electrically controlled three-way valve 73 and 74. The vertical port of the second valve 72 may be communicated with the storage bottle 51. The connections between the vertical ports of the first and second valves 73, 74 and bioreactor 50 may be realized by control of the valves by the direction controller 55. In other words, the connection between the direction controller 55 and reactor 50 is realized by self-shift of the controller 55.

The bioreactor control system formed by the above cycle circuit is mostly used during the period of cell culture by the reactor 50. During period of treatment using the reactor 50, the second fluid is supplied by the patient. The cycle circuit is maintained by change of diastole and systole of the patient heart and therefore, there is no need to provide the direction controller 55 and pump 54. Accordingly, the bioreactor control system and method of the invention are described below in the context of cell culture.

The control system of the invention is related to the bioreactor 50 as described above in the first embodiment. At first, the storage bottle 51 is filled with a culture medium (a second fluid) containing nutrients. The reactor 50 is filled with another culture medium (a first fluid) containing the cells to be cultured. As shown in FIGS. 8-11, one of two conduits coming from the storage bottle 51 is communicated with at least one of the oxygenators 52 and 53 so that oxygenation happens here. Next, the oxygenators 52 and 53 are connected with the pump 54 so as to provide power for circulating the second fluid. After that, the pump 54 is connected with the vertical port of the first fixed electrically controlled three-way valve 71 of the direction controller 55. In addition, the vertical port of the second valve 72 of the controller 55 is coupled directly with the other conduit. Then, the valves 73 and 74 of the controller 55 are connected with the outer inlet 310 and outer outlet 320 of the reactor 50 respectively. A set of predefined parameters is assigned to the controller 55 so that the cycle circuit can be shifted automatically and at a predefined time interval without manual intervention. Obviously, for the reactor 50 of the first embodiment, the outlet path 32 and inlet path 31 are exchangeable with each other, depending upon the direction determined by the controller 55.

Given the direction of FIG. 10, when in work, the culture medium which carries the nutrients is driven by the dynamic pump 54 to move from the storage bottle 51, travels through one of the two conduits and finally reaches the oxygenators 52 and 53 so as to perform oxygenation. After that, the culture medium containing the nutrients and oxygen comes out of the oxygenators 52 and 53, and then flows into the controller 55 under the drive of the pump 54. The controller 55 diverts the second fluid originated from the pump 54 to the outer inlet 310 of the inlet path 31 located at the left side of the reactor. The second fluid comes into the reaction chamber 10 to perform the biochemical reaction with the first fluid. The cells contained in the first fluid absorb the nutrients and oxygen of the second fluid. Then, the second fluid reflowed to the controller 55 through the outer outlet 320 of the outlet path 32 located at the right side. After that, the second fluid is guided into the other conduit of the storage bottle 51 by the controller 55, thereby finishing a complete circulation. The oxygenators 52 and 53 and dynamic pump 54 operate in real time, whereas the direction controller 55 work according to user's setting.

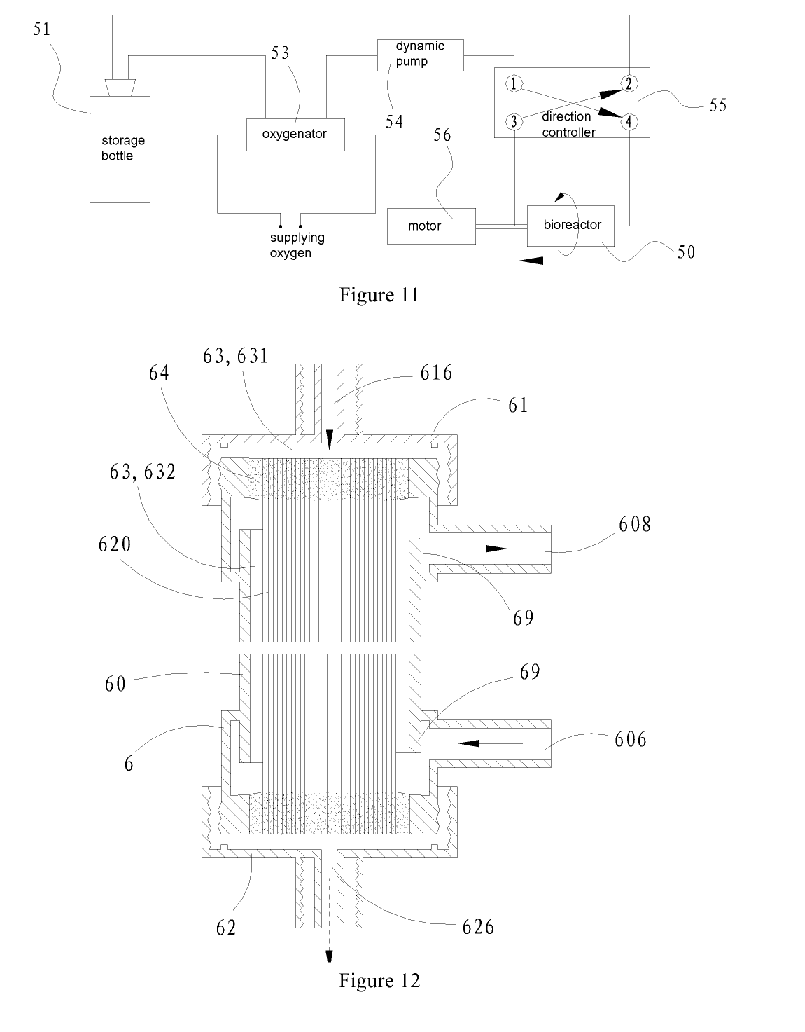

As shown in FIG. 11, after completion of automatic direction change by the controller 55, the second fluid entered under the drive of the pump 54 is guided into the outer inlet 310 of the inlet path 31 located at the right side of the figure. Finally, the second fluid travels to the controller 55 via the outer outlet 320 of the outlet path 32 located at the left side of the figure. Then, the second fluid, to which reaction has been performed, is guided by the controller 55 into the storage bottle 51 through the other conduit, thus finishing a complete circulation.

It is evident from above discussion that in the embodiment of bioreactor control system incorporating the bioreactor of the first embodiment, the outlet path 32 and inlet path 31 of the reactor 50 are exchangeable with each other. As to the interior of the reactor 50, the tying member 400 is provided on the central portion along the longitudinal direction of the mandrel 3 and as a result, bi-directional perfusion is realized inside the reaction chamber 10. Advantageously, bi-directional perfusion makes the cell density at two sides of the chamber 10 consistent with each other and makes the exchange between the two kinds of fluids inside the chamber 10 more uniform.

Of course, unidirectional perfusion is also possible for the control system of the invention, though exchange uniformity of the fluids may be decreased.

It is noted that in FIG. 8 two oxygenators 52 and 53 are employed in the invention.

Referring to FIG. 12, the oxygenator 53 includes a cylinder 6 having a circumferential wall 60 and two end walls 61 and 62. The two end walls 61, 62 are cover elements having internal thread. The external wall of the circumferential wall 60 has external thread formed thereon along the axial direction. Therefore, the two end walls 61 and 62 can be fastened to the two ends of the wall 60 by threaded manner. Of course, regardless of convenience of installation, dismounting and maintenance, at least one of the walls 61 and 62 may be integrally formed with the wall 60.

The two walls 61, 62 and the wall 60 define together inside the cylinder 6 a combination chamber 63 into which a group of hollowed fibers 620 is disposed. Each fiber of the fiber group 620 is arranged to be parallel to the axis of the cylinder 6. In other words, the fiber group 260 is parallel to the axis of the cylinder 6. There is a gap between two adjacent hollowed fibers. The two lateral sides of the fiber group 620 are sealed with the inner wall of the combination chamber 63 of the cylinder 6 by for example adhesive. These fibers are also secured each other at two adhesive locations 64 of the fiber group 620. Part of fibers located between the two adhesive locations 64 along with their gaps defined among these fibers constitutes a fluid flowing chamber 632 which is part of the combination chamber 63. The hollowed chambers of fibers form a gas flowing chamber 631 which is also part of the combination chamber 63. It is well known that the hollowed fiber is tubular; the tube wall of the fiber is air-permeable; but not permeable for the fluid. As such, when the gas passes through the hollowed chamber of each fiber, part of the gas will penetrate the tube wall, while the fluid cannot penetrate the tube wall and enter the hollowed chamber.

The gas flowing chamber 620 defined by the fiber group 620 and the cylinder 60 doesn't overlap the fluid flowing chamber 632. From a transverse cross-sectional view of the cylinder 6, the chamber 632 substantially encloses the chamber 631.

As described above, the gas flowing chamber 631 is used for passage of oxygen, whilst the fluid flowing chamber 632 is intended for passage of the culture medium (the second fluid). Due to permeability feature of the fiber group 620, the fluid is retained inside the fluid flowing chamber 632, and is unable to penetrate the tube wall of the hollowed fiber, thus being prevented from entering the gas flowing chamber 631. The oxygen inside the gas flowing chamber 631, on the contrary, can penetrate the tube wall of the fiber and enter the chamber 632 to combine with the culture medium. Inside the chamber 632, biochemical reaction occurs between the gas and fluid. Furthermore, the gas will not be able to leak out of the cylinder 6 due to good gas sealability of the cylinder 6.

To supply oxygen to the chamber 631, a gas inlet 616 is formed on one end wall 61, and a gas outlet 626 is formed on the other end wall 12. Both the inlet 616 and outlet 626 communicate with the gas flowing chamber 631. A buffer gap is defined between the end wall 61 and a corresponding end portion of the fiber group 620. Another gap is also defined between the end wall 62 and a corresponding end portion of the fiber group 620. The buffer gaps function to buffer the gas entered therein. As the distance between the inlet 616 and outlet 626 is consistent with the longitudinal length of the cylinder 6, once entered the chamber 631, oxygen will have sufficient movement distance to go before it leaves away from the chamber 631. The interface area between the chambers 631 and 632 is enlarged due to existence of gaps among the hollowed fibers. As a result, oxygen has sufficient time and contact area to pass through the fiber group 620 and interact with the fluid contained inside the fluid flowing chamber 632.

Under consideration that the chamber 632 encloses substantially the gas flowing chamber 631 and to supply culture medium fluid to the chamber 632, a fluid inlet 606 and a fluid outlet 608 may be provided on the circumferential wall 60 at any two locations. The inlet 606 and outlet 608 are both communicated with the chamber 632. The fluid is able to enter the chamber 632 through the inlet 606 to interact with oxygen and flow out of the chamber 632 via the outlet 608.

The inlet 606 and outlet 608 are designed to be a straight passage. The fluid entered from the inlet 606 and flowed out through the outlet 608 is driven by a dynamic pump (not shown). The nutrients inside the culture medium and soft fiber group 620 will be damaged by uncontrollable flow velocity, especially when the flow velocity is high. For example, the fiber group 620 might be damaged or deformed by overwhelming impact of the fluid entered along the straight passage. To eliminate this risk, a buffer plate 69 may be mounted in the inlet 606 or outlet 608 for changing the straight passage of the inlet 606 and outlet 608 to non-straight passage. After hitting the buffer plate 69, flow direction of the fluid will be change so that the fluid will flow into the chamber 632 along the peripheral region of the plate 69, thus impact caused by the fluid when entering the chamber 632 being reduced greatly. By this way, the fiber group 620 is effectively protected.

To facilitate manufacture, the buffer plates 69 are disposed on the inlet 606 and 608 at locations close to the wall 60. Further, the buffer plate 69 preferably surrounds the wall 60 and accordingly has a circular shape. Moreover, the space between the wall 60 and buffer plate 69 may be changed to enhance the flow rate of the fluid.

One can expect that the chambers 631 and 632 are exchangeable with each other without departing from the scope of the invention.

Improvement to the oxygenator 53 makes it possible to supply oxygen to the chamber 631 independently by an oxygen supplier. In addition, interaction between oxygen and the second fluid contained inside the chamber 632 occurs in a completely sealed environment and accordingly, no leakage of oxygen could happen. The amount of oxygen may be controlled suitably to ensure the oxygen amount of the second fluid, thus maintaining sufficient supply of the nutrients and oxygen to the cells in the reaction chamber 10.

Another bioreactor control system relevant to the reactor of the second embodiment of the invention is similar to the control system described above except for its direction controller 55. However, it should be noted that in this embodiment the location of the outlet path 32 and inlet path 31 of the reactor 50 is fixed. As shown in FIG. 2, the inlet path 31 is disposed at the right side, while the outlet path 32 is disposed at the left side of the reactor 50. As such, the dynamic pump 54 is needed to connect with the outer inlet 310 of the inlet path 31 at the right side of FIG. 2, while the outer outlet 320 of the outlet path 32 of the reactor 50 is directly coupled with the storage bottle 51. It is because that inside the chamber 10, the tying member 400 is positioned close to the inner inlet 313, and radiation type of flow of the second fluid can be generated only if the second fluid enters the chamber 10 through the inner inlet 313. If the locations of the inlet path 31 and outlet path 32 are changed with each other, then the second fluid, when passing through the tying member 400 at right side from left side, will be slowed down and therefore, can't pass across the tying member 400. Of course, the direction controller 55 may be remained and has the flow direction as shown in FIG. 11. This ensures that the inlet path 31 is at right side, while the outlet path 32 is at the left side of the reactor.

Preferably, the control system related to the third embodiment of the reactor is also unidirectional. As shown in FIG. 6, the path 31 is at the right side, while the path 32 is at the left side of the figure, thus keeping connection as shown in FIG. 11 unchanged. Herein, the direction controller 55 may also be omitted, as long as the dynamic pump 54 has the proper direction.

What is claimed is:

1. A bioreactor, comprising a cylinder, a mandrel and a filter film, wherein the cylinder comprises two end walls and a circumferential wall interconnected with the both end walls; the two end walls and circumferential wall define together a reaction chamber into which reaction happens between a first fluid containing a first kind of substance and a second fluid containing a second kind of substance; the mandrel is disposed between and traverse the two end walls; the mandrel has an inlet path through which the second fluid can enter the reaction chamber and an outlet path through which the second fluid flows out of the reaction chamber provided on its both ends; the filter film wraps the mandrel to prevent passage of the first kind of substance while permit the passage of the second kind of substance; a gap is defined between the filter film and mandrel; at least one portion of the filter film is tied so as to divide the gap into multiple separate and isolated gap regions to prevent the second fluid from direct exiting from the outlet path through the gap.

2. The bioreactor according to claim 1, wherein the tying member is circular, and it tie the filter film and mandrel directly.

3. The bioreactor according to claim 2, wherein the tying member is located at a central location of the mandrel.

4. The bioreactor according to claim 1, wherein the tying member is of a pie shape and has an axle hole; and the tying member surrounds the filter film so as to make the filter film and mandrel tightened with each other.

5. The bioreactor according to claim 4, wherein the tying member is disposed adjacent the inlet path.

6. The bioreactor according to claim 1, wherein there are a number of the tying members located on the filter film to divide the gap into three or more gap regions; the outlet path is communicated with a corresponding gap region so that the second fluid inside the reaction chamber can flow out via said gap region; and the outlet path communicates with at least one of the rest gap regions so that the second fluid can enter the reaction chamber via these gap regions.

7. The bioreactor according to claim 6, wherein the two or more tying members are distributed with an equal distance therebetween.

8. The bioreactor according to claim 1, wherein a mesh barrel is disposed at a location between the filter film and mandrel, of a gap region corresponding to the outlet path; and a plurality of meshes is defined in said mesh barrel.

9. The bioreactor according to claim 1, wherein the cylinder comprises a sample taking opening and a sample supplying opening.

10. A bioreactor control system, comprising:
a bioreactor according to claim 1;
a storage bottle for holding the second fluid containing the second kind of substance;
a dynamic pump for ensuring that the second fluid inside the storage bottle passes through the reaction chamber of the reactor and then returns to the storage bottle so as to form a cycle circuit; and
a motor for driving the bioreactor rotated around its mandrel.

11. The bioreactor control system according to claim 10, further comprising a direction controller for shifting direction of the cycle circuit.

12. The bioreactor control system according to claim 11, further comprising an oxygenator for combine oxygen supplied by an oxygen source with the second fluid inside the cycle circuit.

13. The bioreactor control system according to claim 12, wherein the oxygenator comprises a cylinder having a circumferential wall and two end walls; the two walls and the circumferential wall define together a combination chamber into which a group of hollowed fibers is disposed; the two lateral sides of the fiber group are sealed with the combination chamber so as to form at two adhesive locations a fluid flowing chamber through which the second fluid passes; the hollowed chamber of fibers form a gas flowing chamber through which oxygen passes; the cylinder has a gas inlet and a gas outlet both of which communicate with the gas flowing chamber;
and the cylinder further has a fluid inlet and fluid outlet both communicating with the fluid flowing chamber.

14. The bioreactor control system according to claim 13, wherein the fluid inlet and fluid outlet each have a buffer plate so that the second fluid enters the fluid flowing chamber in a non-straight manner.

15. A control method for a bioreactor according to claim 1, comprising the following steps:
filling in advance the reaction chamber of the bioreactor with a first fluid containing a first kind of substance;
preparing a second fluid containing a second kind of substance;
providing power such that, the second fluid enters the reaction chamber via the inlet path of the reactor, reacts with the first fluid in the reaction chamber, and then reflows via the outlet of the reactor, thus forming a cycle circuit;
providing power such that the reactor rotates around its mandrel, thus making the first and second fluids inside the reaction chamber reacting more uniformly.

16. The bioreactor control method according to claim 15, further comprising the step of combining oxygen with the second fluid inside the cycle circuit.

17. The bioreactor control method according to claim 16, further comprising the step of shifting the direction of the second fluid inside the cycle circuit in a constant interval.
